(19) United States
(12) Patent Application Publication
(10) Pub. No.: US 2005/0277989 A1
(43) Pub. Date: Dec. 15, 2005

(54) Layered deposition braze material

(75) Inventor: Gary D. Schnittgrund, Granada Hills, CA (US)

Correspondence Address: ALFRED E. MANN FOUNDATION FOR;SCIENTIFIC RESEARCH, PO BOX 905, 25134 RYE CANYON LOOP, SUITE 200, SANTA CLARITA, CA, 91380, US

(73) Assignee: Alfred E. Mann Foundation for Scientific Research, Santa Clarita, CA (US)

(21) Appl. No.: 11/178,056

(22) Filed: Jul. 7, 2005

Related U.S. Application Data
(63) Continuation-in-part of application No. 10/793,006, filed on Mar. 3, 2004.

(51) Int. Cl. A61F 2/02; A61N 1/00

(52) U.S. Cl. 607/2; 623/23.72; 623/23.51

(57) ABSTRACT

The invention is a component and method of bonding a ceramic part to a metal part by heating a component assembly comprised of the metal part, the ceramic part, and a thin compact interlayer material placed between the two parts and heated at a temperature that is greater than the temperature of the eutectic formed within the compact interlayer material or between the metal part and the compact interlayer material, but that is less than the melting point of the ceramic part or of the metal part. The component assembly is held in intimate contact at temperature in a non-reactive atmosphere for a sufficient time to develop a strong bond between the ceramic part and the metal part. The compact interlayer material may be further comprised of two or more sets of metal alloy particles each having distinct compositions.

### CROSS REFERENCE TO RELATED APPLICATION

This application is related to but in no way dependent on commonly assigned U.S. Patent Applications, Ceramic to Metal Seal, Attorney Docket No. A366-USA or Ceramic to Metal Seal, Attorney Docket No. A367-USA, both filed on even date herewith and both incorporated herein by reference. This application is a continuation-in-part of co-pending U.S. patent application Ser. No. 10/793,006, filed Mar. 3, 2004.

### FIELD OF THE INVENTION

This invention relates to a method of producing a hermetically sealed ceramic to metal bond for implantation in living tissue.

### BRIEF DESCRIPTION OF THE DRAWINGS

FIG. 1 illustrates a side view of the component assembly with the laminant interlayer material as a foil between the ceramic and metal parts.

FIG. 2 schematically depicts the bonding steps of the present invention.

FIG. 3 illustrates the compact interlayer material comprised of material spheres.

### DETAILED DESCRIPTION OF THE PREFERRED EMBODIMENT

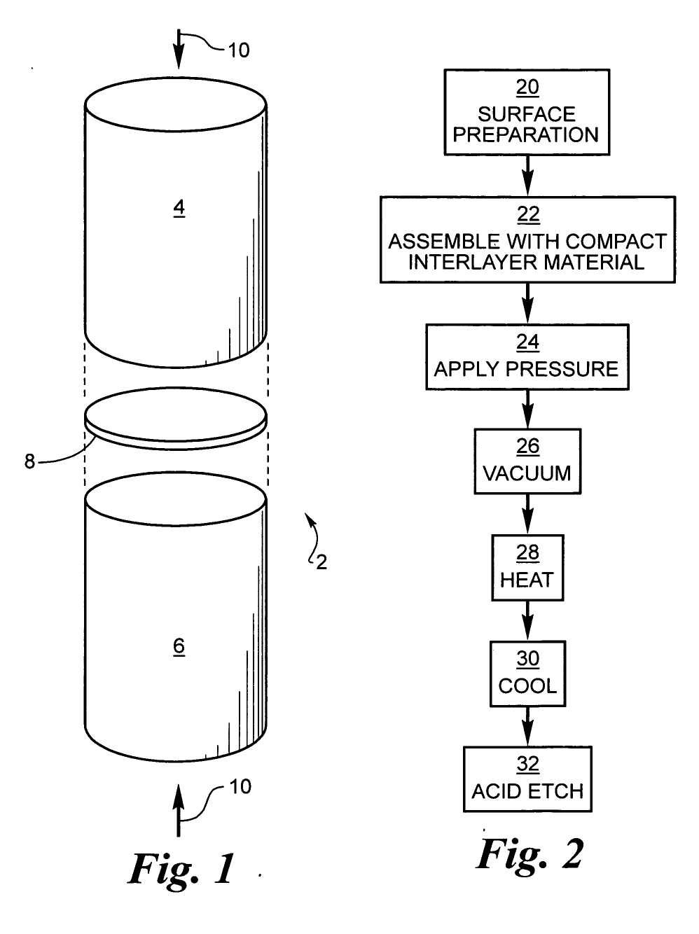

FIG. 1 shows component assembly 2 having metal part 4, ceramic part 6, and compact interlayer material 8. Component assembly 2 is heated to a specific process temperature, which is below the melting point of metal part 4, for a specific period of time, at a pressure that is created by force 10 and that is exerted so as to place compact interlayer material 8 in intimate contact with the metal and ceramic parts.

Compact interlayer material 8 is comprised of a metal compact having a thickness of about five-thousandths of an inch or less (130 μm or less). Compact interlayer material 8 is a bonded compact comprised of at least two sets of metal particles selected from the group of materials that are compatible with the ceramic chosen for ceramic part 6 in that they wet the surface during the bonding process and enter into a diffusion process with the ceramic part 6 thereby creating a strong bond joint during processing. The metal particles comprising compact interlayer material 8 are also selected from the group of materials that are compatible with the metal chosen for a metal part 4. Compact interlayer material 8 forms a bond with the metal part 4 by virtue of developing a eutectic alloy at the bonding temperature and pressure utilized during processing. The lowest eutectic temperature, for example, in the nickel-titanium system is about 942° C. at about 28 weight percent nickel and 72 weight percent titanium. The group of laminated interlayer materials includes substantially pure nickel, i.e., pure nickel and nickel containing approximately two percent or less by weight of other alloy metals and substantially pure titanium, i.e., pure titanium and titanium containing approximately two percent or less by weight of other alloy metals.

Metal part 4 may be selected from a group of biocompatible materials, such as a titanium alloy, and is Ti-6Al-4V in a preferred embodiment. Ceramic part 6 may be alumina, titania, zirconia, stabilized-zirconia, partially-stabilized zirconia, tetragonal zirconia, magnesia-stabilized zirconia, ceria-stabilized zirconia, yttria-stabilized zirconia, and calcia-stabilized zirconia, and in a preferred embodiment ceramic part 6 is yttria-stabilized zirconia. In alternative embodiments, rather than using laminated interlayer material 8 as a foil, interlayer material 8 may be a stack of thin coatings that are applied to either the metal part 4 or ceramic part 6 surface to be bonded by any of a variety of chemical processes such as electroless plating and electroplating, or by any of a variety of thermal processes such as sputtering, evaporating, or ion beam enhanced deposition.

The process steps that are employed to create assembly 2 with a strong bond between metal part 4 and ceramic part 6 are schematically represented in FIG. 2. First, the surfaces to be bonded are prepared in step 20 by machining to assure that they will intimately conform to each other during bonding. The surfaces are smoothed and cleaned.

In step 22, component assembly 2 is prepared with compact interlayer material 8 between metal part 4 and ceramic part 6. In step 24, force 10 is applied to compress laminated interlayer material 8 between metal part 4 and ceramic part 6. Force 10 is sufficient to create intimate contact between the parts. Force 10 is applied to assure that a homogeneous bond is formed between metal part 4 and ceramic part 6, thus creating a hermetic seal between the two parts.

In step 26 the assembly to be heat processed is placed in a furnace in a non-reactive atmosphere, which is preferably vacuum, but which can be argon in an alternative embodiment. A vacuum is applied before the furnace is heated to the processing temperature in step 28. A preliminary holding temperature may be used to allow the thermal mass of the parts to achieve equilibrium before proceeding with heating. The process temperature is lower than the melting point of metal part 4, but greater than the temperature of the eutectic formed by metal 4 and compact interlayer material 8. It is notable that the compact interlayer material 8 behaves significantly differently from an alloy of nickel-titanium when heated, as in the application of a braze foil. It is well known that an alloy of nickel-titanium will behave according to the phase diagram relationships that exist for that alloy composition or that exist for that same alloy composition that is heated in contact with a nickel body. On the other hand, for example, heating a pure nickel material in contact with a pure titanium material results in at least some liquidus formation at the lowest eutectic temperature, which is about 942° C. (at the eutectic composition of about 28 weight percent nickel and 72 weight percent titanium).

In a preferred embodiment, the vacuum is 10⁻⁶ to 10⁻⁷ torr, to assure that the compact interlayer material 8 and metal part 4 do not oxidize. Component assembly 2 is held at the selected temperature, which is typically between approximately 942° and 1080° C., for approximately 5 to 20 minutes, while force 10 continues to be exerted on laminated interlayer material 8. The exact time, temperature and pressure are variable with each other so as to achieve a homogeneous and strong bond of metal part 4 with ceramic part 6. For example, in a preferred embodiment, an yttria-stabilized zirconia part bonds to a Ti-6Al-4V part in vacuum at 10⁻⁶ torr at approximately 980° C. for 10 minutes with a pressure of approximately 5 to 20 psi on a laminated foil comprised of at least one commercially pure nickel layer of approximately 0.0007 inches thickness and at least one titanium layer of 0.0013 inches, yielding a 50 weight percent nickel and 50 weight percent mean composition titanium compact interlayer material 8.

The component assembly 2 is furnace cooled to room temperature in step 30. In optional step 32, component assembly 2 is cleaned by being placed in a bath, after thermal processing is complete, to assure removal of all nickel and nickel salts. This bath is preferably an acid bath that etches the exposed surfaces of component assembly 2. In a preferred embodiment, the bath is nitric acid. Removal of nickel and nickel salts in the bath etch insures that component assembly 2 is biocompatible. Nickel and nickel salts are detrimental to living animal tissue. In a preferred embodiment, however, all of the nickel that is introduced as laminated compact material 8 is combined with the titanium and is combined chemically to be unavailable as free nickel or as a nickel salt.

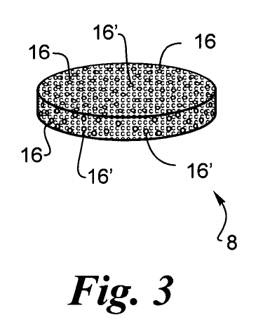

The compact interlayer material 8, illustrated in FIG. 3, is comprised of a multitude of small particles, preferably uniform spheres, each having a controlled composition. For example, spheres of two distinct compositions, primary alloy sphere 16, which may be nickel, and secondary alloy sphere 16′, which may be titanium, are uniformly combined into a homogeneous compact that sinters into a dense compact at temperature and optionally under pressure, as previously illustrated with force 10 of FIG. 1. There may be more than two sets of spheres having different compositions or morphologies, but these alternatives are not illustrated. It is known that the small particles may have other shapes than spheres and that they may not be uniform in size or shape. Further, the final composition of the compact interlayer material 8 is controlled by total volume of primary alloy sphere 16 and secondary alloy sphere 16′ and as the compact interlayer material 8 has been additionally alloyed by diffusion from metal part 4 and/or ceramic part 6 of FIG. 1. It is preferred that the mean composition of the compact interlayer material 8 be chosen so that depletion of either or any of the materials, nickel or titanium, for example, does not occur during the brazing operation. Preferred mean compositions for compact interlayer material 8 contain about 20 to 70 volume percent of nickel with the balance titanium.

It is preferred that the mean composition of the compact interlayer material 8 be chosen so that depletion of a metal, nickel or titanium, for example, does not occur during the brazing operation. Preferred mean compositions for compact interlayer material 8 contain about 20 to 70 volume percent of nickel with the balance titanium. While numerous metals demonstrate formation of eutectic compositions, nickel and titanium are exemplar selections that are useful in an implantable device. Other materials that demonstrate eutectic formation include nickel-titanium, titanium-copper-silver, titanium-copper-nickel, gold-tin, copper-silver, copper-magnesium, copper-titanium, niobium-nickel, nickel-silicon, nickel-zirconium, silver-silicon, silver-tin, silver-titanium, gold-silicon, and gold-titanium.

Component assembly 2 is preferably biocompatible after bonding and processing. Metal part 4, ceramic part 6, and laminated interlayer material 8 are selected to be compatible with the environment in a living body. Hence, metal part 4 is typically a titanium alloy and ceramic part 6 is typically zirconia.

In a preferred embodiment, component assembly 2 is either an electrical sensor or an electrical stimulator that is implanted in a human body, although it could equally well be implanted in any animal. It must survive long periods in the hostile environment of a living body, which is basically a warm saline solution. In a preferred embodiment, component assembly 2 is either a sensor or stimulator comprised of a hollow ceramic tube that contains various electronic components that is bonded to a metal electrode end. The component assembly is preferably watertight; hence, the bond is hermetic, resisting salt-water intrusion as well as growth of living tissue into the metal-to-ceramic bond joint.

Further, component assembly 2 does not corrode while implanted in the body. The materials are chosen such that post-bonding they are not susceptible to corrosion either individually or in the as-bonded state. Component assembly 2 resists electrolytic corrosion as well as crevice corrosion, because of the materials selected for component assembly 2.

Obviously, many modifications and variations of the present invention are possible in light of the above teachings. It is therefore to be understood that, within the scope of the appended claims, the invention may be practiced otherwise than as specifically described.

1. A component assembly for use In living tissue comprising:
a ceramic part;
a metal part; and
a compact interlayer material comprised of at least two sets of metal particles, each set comprised of a metal selected from a group of metals that form a eutectic composition, for bonding said ceramic part to said metal part.

2. The component assembly according to claim 1, wherein said at least two sets of metal particles are comprised of substantially pure metals.

3. The component assembly according to claim 1, wherein said metal particles are approximately spherical in shape.

4. The component assembly according to claim 1, wherein said compact interlayer material is comprised of a set of primary metal particles comprised of nickel particles and a set of secondary metal particles comprised of titanium.

5. The component assembly according to claim 4, wherein said nickel particles and said titanium particles are approximately spherical in shape.

6. The component assembly according to claim 1, wherein said ceramic part is selected from the group consisting of alumina, titania, zirconia, stabilized-zirconia, partially-stabilized zirconia, tetragonal zirconia, magnesia-stabilized zirconia, ceria-stabilized zirconia, calcia-stabilized zirconia, and yttria-stabilized zirconia.

7. The component assembly according to claim 1, wherein said metal part is selected from the group consisting of titanium and titanium alloys.

8. The component assembly according to claim 1, wherein said compact interlayer material reacts with and forms a bond between said ceramic part and said metal part.

9. The component assembly according to claim 1, wherein said assembly is heated to a temperature that is less than the melting point of said metal part but that is greater than the eutectic temperature of said compact interlayer material and said metal part, thereby forming a bond.

10. The component assembly according to claim 1, wherein:
said compact interlayer material having a thickness of approximately 0.005 inches or less; and
said component assembly is heated to a temperature that is less than the melting point of said metal part but that is greater than the eutectic temperature of said compact interlayer material and said metal part, thereby forming a bond.

11. A method of producing a hermetically sealed ceramic to metal bond for implantation in human tissue, the method comprising the steps of:
selecting a ceramic part;
selecting a metal part;
selecting a compact interlayer material;
interposing said interlayer material between said ceramic part and said metal part;
applying a force to said ceramic part and said metal part to place said Interposed interlayer material in compression;
placing assembly of the ceramic and metal parts and Interlayer material in a non-reactive atmosphere;
heating said assembly to a process temperature lower than the melting point of metal part but greater than the eutectic temperature of said compact interlayer material and said metal part;
holding said assembly at said process temperature for a predetermined time to form a bond between said ceramic part and said metal part; and
cooling said assembly.
